(19) United States
(12) Patent Application Publication
(10) Pub. No.: US 2010/0178341 A1
(43) Pub. Date: Jul. 15, 2010

(54) BILAYERED TABLET COMPRISING NIACIN AND HMG-CoA REDUCTASE INHIBITOR

(75) Inventors: Chayapathy ISSA, Madanapalle (IN); Praveen Raheja, New Delhi (IN); Sachin Arora, Muzaffarnagar (IN); Rajesh Gandhi, Gurgaon (IN); Rajeev Shanker Mathur, Gurgaon (IN)

Correspondence Address: Ranbaxy Inc., Intellectual Property Department, 600 College Road East, PRINCETON, NJ, 08540, US

(73) Assignee: RANBAXY LABORATORIES LIMITED, Gurgaon (IN)

(21) Appl. No.: 12/481,832

(22) Filed: Jun. 10, 2009

(30) Foreign Application Priority Data
Jun. 11, 2008 (IN) 1384/DEL/2008

(51) Int. Cl. A61K 9/24; A61K 31/4406; A61P 9/00; A61P 3/06

(52) U.S. Cl. 424/472; 514/356

(57) ABSTRACT

The present invention relates to a bilayered tablet comprising: a slow release layer comprising niacin and release retarding agent; and an immediate release layer comprising HMG-CoA reductase inhibitor.

### FIELD OF THE INVENTION

The present invention relates to a bilayered tablet comprising Niacin and HMG-CoA reductase inhibitor and process of preparation thereof.

### BACKGROUND OF THE INVENTION

Niacin is also known as nicotinic acid. It is chemically, 3-pyridine carboxylic acid, and has been used for many years in the treatment of hyperlipidemia and hypercholesterolemia. This compound has been known to exhibit the beneficial effects of reducing total cholesterol, very low density lipoprotein (VLDL-Cholesterol) and VLDL-cholesterol remnants, LDL-cholesterol, triglycerides and apolipoprotein, while increasing desirable HDL-cholesterol.

A fast release nicotinic acid has conventionally been administered three times per day after meals, but cutaneous flushing often occurs in the hyperlipidemics to whom the nicotinic acid is administered.

In order to avoid or alleviate the cutaneous flushing resulting from nicotinic acid therapy, a number of agents have been suggested. Another method of avoiding or reducing the side effects associated with fast release niacin is the use of extended or sustained release formulations. Extended or sustained release formulations are designed to slowly release the active ingredient from the tablet or capsule, which allows a reduction in dosing frequency as compared to the typical dosing frequency associated with conventional or fast dosage forms. The slow drug release reduces and prolongs blood levels of the drug and, thus, minimizes or lessens the cutaneous flushing side effects that are associated with conventional or fast release niacin products.

HMG-CoA reductase inhibitors have also been used for many years to treat hyperlipidemia. Numbers of HMG-CoA reductase inhibitors are commercially available for the treatment of primary hypercholesterolemia, dysbetalipoproteinemia and homozygous familial hypercholesterolemia. However, HMG-CoA reductase inhibitors are known to induce hepatotoxicity, myopathy and rhabdomyolysis.

Combinations of Niacin and HMG-CoA reductase inhibitors are known in the art in the treatment of hyperlipidemia.

U.S. Pat. No. 5,260,305, assigned to E.R. Squibb and Sons Inc., discloses pharmaceutical combination which includes an inhibitor of the enzyme 3-hydroxy-3-methylglutaryl coenzyme A (HMG CoA) reductase, which is pravastatin and a pharmaceutical which reduces serum cholesterol and/or inhibits cholesterol biosynthesis by a mechanism other than inhibiting production of the enzyme HMG CoA reductase, namely, nicotinic acid (niacin) or related acid. A method for reducing serum cholesterol or inhibiting formation of or treating atherosclerosis using the above combination without causing drug-induced myopathy or rhabdomyolysis, is also disclosed.

U.S. Pat. No. 6,090,830, assigned to Fuisz International Ltd., discloses a pharmaceutical product for oral administration in a unit dosage form for treating hyperlipidemia comprising an immediate release HMG-CoA reductase inhibitor in microparticulate form and a sustained release niacin component in microparticulate form.

U.S. Pat. No. 6,469,035, assigned to Kos Pharmaceuticals, discloses a formulation of niacin and HMG-CoA reductase inhibitor, wherein niacin core is coated with a coating comprising HMG-CoA reductase inhibitor.

WO99/06035, assigned to Kos Pharmaceuticals, discloses oral solid pharmaceutical combinations comprising of: (1) an HMG-CoA reductase inhibitor, (2) nicotinic acid, a nicotinic acid compound or mixtures thereof, and (3) a swelling agent to form a sustained release composition for extended release of the nicotinic acid or nicotinic acid compound or mixtures thereof for nocturnal or evening dosing for reducing serum lipids and increasing HDL-cholesterol. Also, a composition for oral administration during the evening hours to alter serum lipids comprised of nicotinic acid and hydroxypropyl methylcellulose in the form of an extended or sustained release tablet or caplet coated with a coating comprising an HMG-CoA reductase inhibitor in immediate release form is disclosed.

WO2007/069827, assigned to Chong Kun Dang Pharmaceutical Corp., discloses a coated bilayered tablet dosage form comprising: a sustained release layer of niacin together with a sustained release polymer and a fast release layer of HMG-CoA reductase inhibitor, a film or layer forming agent and a plasticizer which is coated on the outer layer of the sustained release layer or embedded on the sustained release layer as a separate layer; wherein the fast release layer should necessarily contain a recrystallization inhibiting agent.

The bilayered tablets of the present invention containing slow release Niacin and immediate release HMG-CoA reductase inhibitor (simvastatin or lovastatin) provides alternate formulations that may be bioequivalent to formulations of SIMCOR® (approved by the U.S. Food and Drug Administration containing Niacin extended release and Simvastatin commercially available in 500/20 mg, 750/20 mg, and 1000/20 mg strengths) and ADVICOR® (approved by the U.S. Food and Drug Administration containing Niacin extended release and Lovastatin commercially available in 500/20 mg, 750/20 mg, 1000/20 mg and 1000/40 mg strengths). Further, the bilayered tablets are convenient in terms of manufacturing process, particularly during scale up, as compared to the coated tablets described in the prior art.

### SUMMARY OF THE INVENTION

According to one embodiment there is provided a bilayered tablet comprising: a slow release layer comprising niacin and one or more release retarding agents; and an immediate release layer comprising HMG-CoA reductase inhibitor.

According to another embodiment there is provided a bilayered tablet comprising: a slow release layer comprising niacin, 5-50% w/w of one or more release retarding agents and one or more pharmaceutically acceptable excipients; and an immediate release layer comprising HMG-CoA reductase inhibitor and one or more pharmaceutically acceptable excipients.

According to still another embodiment there is provided a bilayered tablet comprising: a slow release layer comprising niacin, 10-30% w/w of hydroxypropyl cellulose and one or more pharmaceutically acceptable excipients; and an immediate release layer comprising HMG-CoA reductase inhibitor and one or more pharmaceutically acceptable excipients.

According to another embodiment there is provided a bilayered tablet comprising: a slow release layer comprising niacin, 10-30% w/w of polyethylene oxide and one or more pharmaceutically acceptable excipients; and an immediate release layer comprising HMG-CoA reductase inhibitor and one or more pharmaceutically acceptable excipients.

According to one more embodiment there is provided a bilayered tablet comprising: a slow release layer comprising niacin, 10-30% w/w of hydroxypropyl methylcellulose and one or more pharmaceutically acceptable excipients; and an immediate release layer comprising HMG-CoA reductase inhibitor and one or more pharmaceutically acceptable excipients.

According to still another embodiment there is provided a process for preparing the bilayered tablets disclosed in the various embodiments of the specification.

### DETAILED DESCRIPTION OF THE INVENTION

According to one embodiment, the bilayered tablet comprises: a slow release layer of niacin and an immediate release layer of HMG-CoA reductase inhibitor.

The term ‘Niacin’ refers to nicotinic acid compound or any mixtures thereof, and specifically includes, but are not limited to the following: nicotinic acid, nicotinyl alcohol tartrate, D-glucitol hexanicotinate, aluminium nicotinate, niceritrol , D-L-alpha-tocopheryl nicotinate, 6-OH-nicotinic acid, nicotinuaria acid, nicotinamide, nicotinamide-N-oxide, 6-OH-nicotinamide, NAD, N-methyl-2-pyridine-8-carboxamide, N-methyl-nicotinamide, N-ribosyl-2-pyridone-5-carboxide, N-methyl-4-pyridone-5-carboxamide, bradilian, sorbinicate, hexanicite, ronitol, and esters of nicotinic acid such as lower alcohol esters like methyl, ethyl, propyl or butyl esters. Each of such derivatives or compounds will be collectively referred to hereinafter by ‘Niacin’. The amount of niacin may range from about 250 mg to about 3000 mg, for example—from about 500 mg to about 2500 mg. Niacin may be daily dosed in increments of, for example, 250 mg, 500 mg, 750 mg, 1000 mg, 1500 mg, 2000 mg, 2500 mg and 3000 mg. The bilayered tablets of the present invention may include niacin in dosage amounts of, for example—250 mg, 375 mg, 500 mg, 750 mg and 1000 mg.

The ‘HMG-CoA reductase inhibitors’ include, but are not limited to simvastatin, lovastatin, pravastatin free bases or pharmaceutically acceptable salts, solvates or mixtures thereof. The amount of HMG-CoA reductase inhibitor may range from about 0.05 mg to about 160 mg, for example from 5 mg to 80 mg. The exact dosing of HMG-CoA reductase inhibitor may depend upon the particular HMG-CoA reductase inhibitor selected e.g. lovastatin in dosage amounts between about 10 mg and about 80 mg or more, such as 10 mg, 20 mg, 40 mg or 80 mg, simvastatin in dosage amounts between about 5 mg and about 80 mg or more, such as 5 mg, 10 mg, 20 mg, 40 mg or 80 mg.

The bilayered tablet of the present invention include niacin/HMG-CoA reductase inhibitor in dosage strengths of, for example, 250/5 mg, 500/5 mg, 750/5 mg, 1000/5 mg, 250/10 mg, 500/10 mg, 750/10 mg, 1000/10 mg, 250/20 mg, 500/20 mg, 750/20 mg, 1000/20 mg, 250/40 mg, 500/40 mg, 750/40 mg, and 1000/40 mg.

The slow release layer comprises a ‘release retarding agent’ selected from one or more of cellulose derivatives such as hydroxypropyl methylcellulose, hydroxyethyl cellulose, hydroxypropyl cellulose, carboxymethyl cellulose and sodium carboxy methyl cellulose; gums such as xanthan gum, karaya gum, locust bean gum, alginic acid and sodium alginate; vinyl alcohol or vinylpyrrolidone based polymers such as polyvinyl alcohol, polyvinylpyrrolidone; alkylene oxide polymers such as polyethylene oxide and mixtures thereof. A specific grade of hydroxypropyl cellulose e.g. HPC-H, HPC-M, HPC-HX, HPC-HXF by Hercules Inc. and/or polyethylene oxide e.g. PEO-27, PEO-18, PEO-15, PEO-8, PEO-4 by Sumitomo Seika Chemicals Co., Polyox WSR 1105, Polyox WSR 303 by Dow Chemicals, providing desired release profile may be used. The hydroxypropyl methylcellulose may be the commercially available products such as Methocel®. Premium product grades having specific apparent viscosities, e.g., viscosities ranging from about 100-150,000 cP (2% in water at 20° C.) such as K100, K4M, K15M, K100M, E4M, E10M; viscosities ranging from 80000-120,000 cP (2% in water at 20° C.) such as Methocel K100M CR. The release retarding agent is present in an amount of 5-50% by weight of the total formulation. For example, 10-30% w/w of hydroxypropyl cellulose, hydroxypropyl methylcellulose, polyethylene oxide or mixtures thereof.

As used herein, the term ‘immediate release’ shall mean that the release of the majority of the active material content occurs within a relatively short time, for example within 1 hour, preferably within 30 minutes, after oral ingestion.

As used herein, the term ‘slow release’ refers to the release of a drug substance from a pharmaceutical formulation, at a slower rate than from an immediate release formulation.

Both the slow release layer and the immediate release layer further comprise one or more pharmaceutically acceptable excipients.

The ‘pharmaceutically acceptable excipients’ may be selected from one or more of diluents, binders, disintegrants, anti-oxidants, lubricants, glidants and coloring agents.

The ‘diluent’ may be selected from one or more of saccharides like lactose, sucrose and glucose; sugar alcohols like mannitol, sorbitol, xylitol and maltitol.

The ‘binder’ may be selected from one or more of starches like starch, pregelatinized starch and modified starch; cellulose derivatives like hydroxypropyl methyl cellulose, hydroxypropyl cellulose and methyl cellulose; gums like xanthan gum, gum acacia and tragacanth; and water soluble vinylpyrrolidone polymers like polyvinyl pyrrolidone, copolymer of vinyl pyrrolidone and vinyl acetate.

The ‘disintegrant’ may be selected from one or more of crospovidone, hydroxypropyl cellulose, pregelatinized starch, sodium starch glycolate and croscarmellose sodium.

The ‘anti-oxidant’ may be selected from one or more of ascorbic acid, butylated hydroxy anisole, citric acid, and tocopherol.

The ‘lubricant’ may be selected from one or more of talc, stearic acid, colloidal silicon dioxide, magnesium stearate and sodium stearyl fumarate.

The bilayered tablets of the invention may comprise a non-functional coating.

The ‘non-functional coating’ may comprise polymers like hydroxypropyl cellulose, hydroxyethyl cellulose, hydroxypropyl methylcellulose, polyvinyl pyrrolidone, copolymer of vinyl pyrrolidone and vinyl acetate; plasticizers like polyethylene glycol, triacetin, dibutyl sebecate and diethyl tartrate; opacifying agents like titanium dioxide and talc; and coloring agents. Examples of such non-functional coat are commercially available Opadry® compositions.

The bilayered tablet of the invention may be prepared by wet granulation, dry granulation or direct compression process. The wet granulation process involves use of water or any other suitable solvent. The dry granulation may involve use of roller compacter or any suitable technique.

According to one embodiment of the specification there is provided a process for the preparation of the bilayered tablet, the process comprising the steps of:
a) preparing slow release granules comprising niacin, one or more release retarding agents and one or more pharmaceutically acceptable excipients;
b) preparing immediate release granules comprising HMG-CoA reductase inhibitor and one or more pharmaceutically acceptable excipients; and,
c) compressing the granules of steps (a), and (b) into two separate layers.

The following non-limiting examples further illustrate the bilayered tablet of Niacin and HMG-CoA reductase inhibitor and process of making such formulation:

### EXAMPLE 1

| | 500/20 mg | 750/20 mg | 1000/20 mg |
|---|---|---|---|
| Ingredient | Mg/tab | Mg/tab | Mg/tab |
| Slow release layer | | | |
| Intragranular | | | |
| Niacin | 500 | 750 | 1000 |
| Hydroxypropylcellulose | 185 | 184 | 170 |
| (HPC-H) | | | |
| Polyvinylpyrrolidone | 17 | 26 | 34 |
| Purified water | Q.S. | Q.S. | Q.S. |
| Extra granular | | | |
| Hydroxypropylcellulose | 28 | 38 | 34 |
| (HPC-H) | | | |
| Stearic Acid | 8 | 10.0 | 12 |
| Total | 738 | 1008 | 1250 |
| Immediate release Layer | | | |
| Intragranular | | | |
| Simvastatin | 20 | 20 | 20 |
| Lactose Monohydrate | 146.92 | 146.92 | 146.92 |
| Pregelatinized starch | 6.00 | 6.00 | 6.00 |
| Ascorbic acid | 5.0 | 5.0 | 5.0 |
| Microcrystalline cellulose | 12.5 | 12.5 | 12.5 |
| Purified water | q.s. | q.s. | q.s. |
| Butylated Hydroxy Anisole | 0.08 | 0.08 | 0.08 |
| Isopropyl alcohol | q.s. | q.s. | q.s. |
| Extragranular | | | |
| Croscarmellose sodium | 8 | 8 | 8 |
| Magnesium stearate | 1.5 | 1.5 | 1.5 |
| Total | 200 | 200 | 200 |
| Coating | | | |
| Opadry | 28.0 | 36.0 | 43.5 |

### EXAMPLE 2

| | 500/20 mg | 750/20 mg | 1000/20 mg |
|---|---|---|---|
| Ingredient | Mg/tab | Mg/tab | Mg/tab |
| Slow release layer | | | |
| Intragranular | | | |
| Niacin | 500 | 750 | 1000 |
| Polyethylene oxide | 78 | 95 | 120 |
| Polyvinylpyrrolidone | 17 | 26 | 34 |
| Purified water | Q.S. | Q.S. | Q.S. |
| Extra granular | | | |
| Polyethylene oxide | 128 | 119 | 84 |
| Stearic Acid | 7 | 10.0 | 12 |
| Total | 730 | 1000 | 1250 |
| Immediate release layer | | | |
| Intragranular | | | |
| Simvastatin | 20 | 20 | 20 |
| Lactose Monohydrate | 146.92 | 146.92 | 146.92 |
| Pregelatinized starch | 6.00 | 6.00 | 6.00 |
| Ascorbic acid | 5.0 | 5.0 | 5.0 |
| Microcrystalline cellulose | 12.5 | 12.5 | 12.5 |
| Purified water | q.s. | q.s. | q.s. |
| Butylated Hydroxy Anisole | 0.08 | 0.08 | 0.08 |
| Isopropyl alcohol | q.s. | q.s. | q.s. |
| Extragranular | | | |
| Croscarmellose sodium | 8 | 8 | 8 |
| Magnesium stearate | 1.5 | 1.5 | 1.5 |
| Total | 200 | 200 | 200 |
| Coating | | | |
| Opadry | 28.0 | 36.0 | 43.5 |

Brief Manufacturing Process:

A. Slow Release Layer
1. Niacin along with hydroxypropylcellulose or polyethylene oxide and polyvinylpyrollidone was sifted through BSS#30 sieve.
2. The material of step 1 was granulated in rapid mixer granulator using purified water.
3. The granules of step 2 were dried in fluid bed dryer at 50-60° C.
4. The dried granules of step 3 were sifted through BSS#25 sieve.
5. The extragranular hydroxypropylcellulose or polyethylene oxide was sifted through BSS#30 sieve and was blended with the granules of step 4.
6. The extra granular stearic acid was sifted through BSS#30 sieve and was blended with the material of step 5.

B. Immediate Release Layer
7. Simvastatin, Lactose monohydrate, pregelatinized starch, microcrystalline cellulose were sifted through BSS#25 sieve.
8. Butylated hydroxy anisole was dissolved in isopropyl alcohol.
9. Ascorbic acid was dissolved in purified water.
10. The material of step 7 was granulated using the solutions of step 8 and 9 in rapid mixer granulator.
11. The granules of step 10 were dried in fluid bed drier at 50-60° C.
12. The dried granules of step 11 were sifted through BSS#25 sieve.
13. The extra granular croscarmellose sodium was sifted through BSS#30 sieve and was blended with the granules of step 12.
14. The extra granular magnesium stearate was sifted through BSS#30 sieve and was blended with the material of step 13.

Compression
15. The blends of step 6 and step 14 were compressed into bilayered tablets.

Coating
16. Opadry was dispersed in purified water.
17. The bilayered tablets of step 15 were coated using the dispersion of step 16 in a coating pan.

### EXAMPLE 3

| | 500/20 mg | 750/20 mg | 1000/20 mg | 1000/40 mg |
|---|---|---|---|---|
| Ingredient | Mg/tab | Mg/tab | Mg/tab | Mg/tab |
| Slow release layer | | | | |
| Intragranular | | | | |
| Niacin | 500 | 750 | 1000 | 1000 |
| Hydroxypropylcellulose | 185 | 184 | 170 | 170 |
| (HPC-H) | | | | |
| Polyvinylpyrrolidone | 17 | 26 | 34 | 34 |
| Purified water | Q.S. | Q.S. | Q.S. | Q.S. |
| Extra granular | | | | |
| Hydroxypropylcellulose | 28 | 38 | 34 | 34 |
| (HPC-H) | | | | |
| Stearic Acid | 8 | 10.0 | 12 | 12 |
| Total | 738 | 1008 | 1250 | 1250 |
| Immediate release Layer | | | | |
| Intragranular | | | | |
| Lovastatin | 20 | 20 | 20 | 40 |
| Starch | 4.0 | 4.0 | 4.0 | 8.0 |
| Tocopherol | 0.2 | 0.2 | 0.2 | 0.4 |
| Isopropyl alcohol | q.s. | q.s. | q.s. | q.s. |
| Extragranular | | | | |
| Starch | 6.0 | 6.0 | 6.0 | 2.0 |
| Lactose Monohydrate | 130.0 | 130.0 | 130.0 | 114.0 |
| Microcrystalline cellulose | 36.75 | 36.75 | 36.75 | 32.55 |
| Colloidal Silicon dioxide | 2.0 | 2.0 | 2.0 | 2.0 |
| Magnesium stearate | 1.0 | 1.0 | 1.0 | 1.0 |
| FD&C Blue No. 1 Aluminum | 0.05 | 0.05 | 0.05 | 0.05 |
| Lake | | | | |
| Total | 200 | 200 | 200 | 200 |
| Coating | | | | |
| Opadry | 28.0 | 36.0 | 43.5 | 43.5 |

### EXAMPLE 4

| | 500/20 mg | 750/20 mg | 1000/20 mg | 1000/40 mg |
|---|---|---|---|---|
| Ingredient | Mg/tab | Mg/tab | Mg/tab | Mg/tab |
| Slow release layer | | | | |
| Intragranular | | | | |
| Niacin | 500 | 750 | 1000 | 1000 |
| Polyethylene oxide | 78 | 95 | 120 | 120 |
| Polyvinylpyrrolidone | 17 | 26 | 34 | 34 |
| Purified water | q.s. | q.s. | q.s. | q.s. |
| Extra granular | | | | |
| Polyethylene oxide | 128 | 119 | 84 | 84 |
| Stearic Acid | 7 | 10.0 | 12 | 12 |
| Total | 730 | 1000 | 1250 | 1250 |
| Immediate release layer | | | | |
| Intragranular | | | | |
| Lovastatin | 20 | 20 | 20 | 40.0 |
| Starch | 4.0 | 4.0 | 4.0 | 8.0 |
| Tocopherol | 0.2 | 0.2 | 0.2 | 0.4 |
| Isopropyl alcohol | q.s. | q.s. | q.s. | q.s. |
| Extragranular | | | | |
| Starch | 6.0 | 6.0 | 6.0 | 2.0 |
| Lactose Monohydrate | 130.0 | 130.0 | 130.0 | 114.0 |
| Microcrystalline cellulose | 36.75 | 36.75 | 36.75 | 32.55 |
| Colloidal Silicon dioxide | 2.0 | 2.0 | 2.0 | 2.0 |
| Magnesium stearate | 1.0 | 1.0 | 1.0 | 1.0 |
| FD&C Blue No. 1 Aluminum | 0.05 | 0.05 | 0.05 | 0.05 |
| Lake | | | | |
| Total | 200 | 200 | 200 | 200 |
| Coating | | | | |
| Opadry | 28.14 | 36.24 | 43.5 | 43.5 |

Brief Manufacturing Process:

A. Slow Release Layer
1. Niacin along with hydroxypropyl cellulose or polyethylene oxide and polyvinylpyrollidone was sifted through BSS#30 sieve.
2. The material of step 1 was granulated in rapid mixer granulator using purified water.
3. The granules of step 2 were dried in fluid bed dryer at 50-60° C.
4. The dried granules of step 3 were sifted through BSS#25 sieve.
5. The extragranular hydroxypropyl cellulose or polyethylene oxide was sifted through BSS#30 sieve and was blended with the granules of step 4.
6. The extra granular stearic acid was sifted through BSS#30 sieve and was blended with the material of step 5.

B. Immediate Release Layer
7. Lovastatin was sifted through BS S#18 sieve.
8. Starch was sifted through BSS# 45 sieve.
9. Tocopherol was dissolved in isopropyl alcohol.
10. The material of step 7 was granulated in rapid mixer granulator using the solution of step 9.
11. The material of step 8 was added to step 10 and mixed.
12. The material of step 11 was dried in tray dryer at NMT 50° C.
13. FD&C Blue No.1 was sifted through BSS # 100 and was blended with material of step 12.
14. The extra granular starch was sifted through BSS# 45 sieve and was blended with the material of step 13.
15. The extra granular lactose monohydrate, microcrystalline cellulose and colloidal silicon dioxide were sifted through BSS#45 sieve and were blended with the material of step 14.
16. The extra granular magnesium stearate was sifted through BSS#60 sieve and was blended with the material of step 15.

C. Compression
17. The blends of step 6 and step 16 were compressed into bilayered tablets.

D. Coating
18. Opadry was dispersed in purified water.
19. The bilayered tablets of step 17 were coated using the dispersion of step 18 in a coating pan.

### EXAMPLE 5

| | 500/20 mg | 750/20 mg | 1000/20 mg |
|---|---|---|---|
| Ingredient | Mg/tab | Mg/tab | Mg/tab |
| Slow release layer | | | |
| Intragranular | | | |
| Niacin | 500 | 750 | 1000 |
| Hydroxypropyl | 57.5 | 86.25 | 115 |
| methylcellulose (Methocel | | | |
| E10M/K15MCR) | | | |
| Purified water | Q.S. | Q.S. | Q.S. |
| Extra granular | | | |
| Hydroxypropyl | 21 | 31.5 | 42 |
| methylcellulose(Methocel | | | |
| E10M/K15MCR) | | | |
| Stearic Acid | 6 | 9 | 12 |
| Total | 584.5 | 876.75 | 1169 |
| Immediate release layer | | | |
| Intragranular | | | |
| Simvastatin | 20 | 20 | 20 |
| Lactose | 139.02 | 139.02 | 139.02 |
| Starch | 6.00 | 6.00 | 6.00 |
| Ascorbic acid | 5.00 | 5.00 | 5.00 |
| Hydroxypropyl | 25.00 | 25.00 | 25.00 |
| methylcellulose 15 cps | | | |
| Butylated Hydroxy Anisole | 0.08 | 0.08 | 0.08 |
| FD&C Blue#2 | 0.2 | 0.2 | 0.2 |
| Purified water | q.s | q.s | q.s |
| Isopropyl alcohol | q.s | q.s | q.s |
| Extragranular | | | |
| Croscarmellose sodium | 2.5 | 2.5 | 2.5 |
| Magnesium Stearate | 2 | 2 | 2 |
| FD&C Blue#2 | 0.2 | 0.2 | 0.2 |
| Total | 200 | 200 | 200 |
| Tablet coating | | | |
| Opadry | 35.62 | 35.62 | 35.62 |

### EXAMPLE 6

| | 500/20 mg | 750/20 mg | 1000/20 mg |
|---|---|---|---|
| Ingredient | Mg/tab | Mg/tab | Mg/tab |
| Slow release layer | | | |
| Intragranular | | | |
| Niacin | 500 | 750 | 1000 |
| Hydroxypropyl | 75 | 112.5 | 150 |
| methylcellulose(Methocel | | | |
| E10M/K15MCR) | | | |
| Purified water | Q.S. | Q.S. | Q.S. |
| Extra granular | | | |
| Hydroxypropyl | 30 | 45 | 60 |
| methylcellulose(Methocel | | | |
| E10M/K15MCR) | | | |
| Stearic Acid | 6 | 9 | 12 |
| Total | 611 | 916.5 | 1222 |
| Immediate release layer | | | |
| Intragranular | | | |
| Simvastatin | 20 | 20 | 20 |
| Lactose | 139.02 | 139.02 | 139.02 |
| Starch | 6.00 | 6.00 | 6.00 |
| Ascorbic acid | 5.00 | 5.00 | 5.00 |
| Hydroxypropyl | 25.00 | 25.00 | 25.00 |
| methylcellulose 15 cps | | | |
| Butylated Hydroxy Anisole | 0.08 | 0.08 | 0.08 |
| Purified water | q.s | q.s | q.s |
| Isopropyl alcohol | q.s | q.s | q.s |
| Extragranular | | | |
| Croscarmellose sodium | 2.5 | 2.5 | 2.5 |
| Magnesium Stearate | 2 | 2 | 2 |
| FD&C Blue#2 | 0.2 | 0.2 | 0.2 |
| Total | 200 | 200 | 200 |
| Tablet coating | | | |
| Opadry | 35.62 | 35.62 | 35.62 |

Brief Manufacturing Process:

A. Slow Release Layer
1. Niacin along with hydroxypropyl methylcellulose was sifted through BSS#30 sieve
2. The material of step 1 was granulated in rapid mixer granulator using purified water
3. The granules of step 2 were dried in fluid bed dryer at 50-60° C.
4. The dried granules of step 3 were sifted through BSS#22 sieve
5. The extragranular hydroxypropyl methylcellulose was sifted through BSS#30 sieve and was blended with the granules of step 4
6. The extra granular stearic acid was sifted through BSS#44 sieve and was blended with the material of step 5.

B. Immediate Release Layer
7. Simvastatin, Lactose monohydrate, starch, FD&C Blue#2 and hydroxypropyl methylcellulose were sifted through BSS#25 sieve
8. Butylated hydroxy anisole was dissolved in isopropyl alcohol.
9. Ascorbic acid was dissolved in purified water.
10. The material of step 7 was granulated using the solutions of step 8 and 9 in rapid mixer granulator.
11. The granules of step 10 were dried in fluid bed dryer at 50-60° C.
12. The dried granules of step 11 were sifted through Quadro co-mill.
13. The extra granular magnesium stearate was sifted through BSS#30 sieve and was blended with the material of step 12

Compression
14. The blends of step 6 and step 13 were compressed into bilayer tablets.

Coating
15. Opadry was dispersed in purified water.
16. The bilayered tablets of step 14 were coated using the dispersion of step 15 in coating pan.

We claim:

1. A bilayered tablet comprising:
a slow release layer comprising niacin, one or more release retarding agents and one or more pharmaceutically acceptable excipients; and,
an immediate release layer comprising HMG-CoA reductase inhibitor and one or more pharmaceutically acceptable excipients.

2. The tablet of claim 1, wherein the HMG-CoA reductase inhibitor is selected from one or more of simvastatin, lovastatin or pravastatin.

3. The tablet of claim 1, wherein the slow release layer comprises 5-50% w/w of the release retarding agent.

4. The tablet of claim 1, wherein the release retarding agent is selected from one or more of cellulose derivatives, gums, vinyl alcohol or vinylpyrrolidone based polymers, alkylene oxide polymers or mixtures thereof.

5. The tablet of claim 4, wherein the cellulose derivatives are selected from hydroxypropyl methylcellulose, hydroxyethyl cellulose, hydroxypropyl cellulose, carboxymethyl cellulose and sodium carboxymethyl cellulose; gums are selected from xanthan gum, karaya gum, locust bean gum, alginic acid and sodium alginate; vinyl alcohol or vinylpyrrolidone based polymers are selected from polyvinyl alcohol, polyvinylpyrrolidone; alkylene oxide polymers are selected from polyethylene oxide or mixtures thereof.

6. The tablet of claim 1, wherein the pharmaceutically acceptable excipients comprise one or more of diluents, binders, disintegrants, anti-oxidants, lubricants, glidants and coloring agents.

7. The tablet of claim 1, wherein the slow release layer comprises niacin, 10-30% w/w of hydroxypropyl cellulose and one or more pharmaceutically acceptable excipients; and, the immediate release layer comprises HMG-CoA reductase inhibitor and one or more pharmaceutically acceptable excipients.

8. The tablet of claim 1, wherein the slow release layer comprises niacin, 10-30% w/w of polyethylene oxide and one or more pharmaceutically acceptable excipients; and, the immediate release layer comprises HMG-CoA reductase inhibitor and one or more pharmaceutically acceptable excipients.

9. The tablet of claim 1, wherein the slow release layer comprises niacin, 10-30% w/w of hydroxypropyl methylcellulose and one or more pharmaceutically acceptable excipients; and, the immediate release layer comprises HMG-CoA reductase inhibitor and one or more pharmaceutically acceptable excipients.

10. A process for the preparation of the bilayered tablet of claim 1, the process comprising the steps of:
a) preparing slow release granules comprising niacin, one or more release retarding agents and one or more pharmaceutically acceptable excipients;
b) preparing immediate release granules comprising HMG-CoA reductase inhibitor and one or more pharmaceutically acceptable excipients; and,
c) compressing the granules of steps (a), and (b) into two separate layers.
